(19) United States
(12) Patent Application Publication
(10) Pub. No.: US 2025/0367058 A1
(43) Pub. Date: Dec. 4, 2025

(54) FIRE-DRAGON CUPPING DEVICE INTEGRATED WITH FUNCTIONS OF CUPPING AND SCRAPING

(71) Applicant: The Affilliated Hospital of Hunan Academy of Chinese Medicine, Changsha (CN)

(72) Inventors: Haili Zhu, Changsha (CN); Yuxing Guo, Changsha (CN); Ji Luo, Changsha (CN); Baihui Liang, Changsha (CN); Sen Fang, Changsha (CN)

(21) Appl. No.: 19/226,153

(22) Filed: Jun. 3, 2025

(30) Foreign Application Priority Data
Jun. 4, 2024 (CN) 202410715320.9

(51) Int. Cl. A61H 9/00; A61H 7/00; A61H 39/06

(52) U.S. Cl. CPC A61H 9/0057; A61H 7/005; A61H 39/06; A61H 2201/0153; A61H 2201/0207; A61H 2201/0221; A61H 2201/1207; A61H 2201/1664

(57) ABSTRACT

A fire-dragon cupping device integrated with functions of cupping and scraping includes: an outer cupping body; a lotus-petal-shaped mouth structure disposed on the outer cupping body; a circular mouth structure including an inner cupping body and a circular mouth connected to a top of the inner cupping body; a connecting ring disposed on an inner wall of the transmission sleeve; meshing transmission assemblies annularly arranged on a bottom of an interior of the outer cupping body; and a screw rod drive assembly disposed on a bottom of the outer cupping body. The lotus-petal-shaped mouth structure includes: an assembly ring fixedly installed on a top of the outer cupping body, a transmission sleeve, and a lotus-shaped mouth disposed on a top of the transmission sleeve. The fire-dragon cupping device, through position switching of the lotus-petal-shaped mouth structure and the circular mouth structure, possess multiple therapeutic effects of scraping, moxibustion, and cupping.

### CROSS-REFERENCE TO RELATED APPLICATION

This application claims priority to Chinese Patent Application No. 202410715320.9, filed on Jun. 4, 2024, which is herein incorporated by reference in its entirety.

### TECHNICAL FIELD

The disclosure relates to the technical field of fire-dragon cupping devices, and more particularly to a fire-dragon cupping device integrated with functions of cupping and scraping (also known as gua sha, a traditional Chinese medicine therapy).

### BACKGROUND

Both scraping and cupping are therapeutic methods of traditional Chinese medicine, and have functions such as dispelling wind, dispersing cold, removing dampness, reducing swelling and relieving pain, freeing sinews (also referred to as relaxing tendons) and activating collaterals, and activating blood circulation and resolving stasis. Research indicates that the scraping can play a role in activating blood circulation and resolving stasis, and freeing sinews and activating collaterals, while the cupping is more effective in dispelling wind and eliminating dampness. The combination of the scraping and the cupping is conducive to enhancing the expulsion of cold pathogenic qi (also referred to as cold evil-qi, or cold pathogenic factors in traditional Chinese medicine) from the body, thereby achieving therapeutic goals.

Current fire-dragon cupping devices are made of different-sized cupping bodies obtained by mixing and firing bian stone (also referred to as stone needle, which is made of stone used as a primitive instrument for acupuncture) and zisha (also referred to as purple clay), and can be inserted with corresponding moxa cones therein. The fire-dragon cupping devices use a specially customized cupping body and a uniquely designed mouth for gliding cupping. However, due to the lotus-petal-shaped design of its mouth, the fire-dragon cupping device cannot be closely attached to the skin and therefore cannot complete a cupping process. For the consideration of energy saving and practicality, a multifunctional fire-dragon cupping device that can be used for both cupping and scraping has been designed in the disclosure.

### SUMMARY

A purpose of the disclosure is to provide a fire-dragon cupping device integrated with functions of cupping and scraping. The fire-dragon cupping device achieves multiple therapeutic functions including the scraping, moxibustion, and the cupping by position switching between the lotus-petal-shaped mouth structure and the circular mouth structure, and combining the characteristics of both structures. The design concept of the fire-dragon cupping device, which allows for multiple uses with a single pot, not only enhances the therapeutic effects but also improves the practicality of the fire-dragon cupping device.

In order to achieve the above purpose, the disclosure provides following technical solutions. The fire-dragon cupping device integrated with functions of cupping and scraping includes: an outer cupping body, a lotus-petal-shaped mouth structure disposed on the outer cupping body, a circular mouth structure, a connecting ring, meshing transmission assemblies, and a screw rod drive assembly. The lotus-petal-shaped mouth structure includes an assembly ring, a transmission sleeve, first limit strips, and lotus-shaped mouth. The assembly ring is fixedly mounted on a top of the outer cupping body, the first limit strips are disposed around the transmission sleeve and movable up and down in first limit grooves defined on an inner wall of the assembly ring respectively, the lotus-shaped mouth is disposed on a top of the transmission sleeve, and the lotus-petal-shaped mouth structure is configured to perform scraping and moxibustion operations of the fire-dragon cupping device. The circular mouth structure includes an inner cupping body and a circular mouth, the circular mouth is connected to a top of the inner cupping body, and the circular mouth structure is configured to improve airtightness when the fire-dragon cupping device is inverted and pressed to thereby perform a cupping operation through vacuum suction. The connecting ring is disposed on an inner wall of the transmission sleeve, the inner cupping body is provided with second limit strips arranged therearound, and the second limit strips are movable up and down in second limit grooves defined on an inner wall of the connecting ring respectively. Meshing transmission assemblies are annularly arranged on a bottom of an interior of the outer cupping body, and each of the meshing transmission assemblies is disposed between the lotus-petal-shaped mouth structure and the circular mouth structure and configured to automatically switch vertical positions of the lotus-petal-shaped mouth structure and the circular mouth structure. The screw rod drive assembly is disposed on a bottom of the outer cupping body, and the screw rod drive assembly is configured to drive the circular mouth structure to move downwards when performing the scraping and moxibustion operations, and correspondingly the lotus-petal-shaped mouth structure automatically moves upwards through meshing engagement, and configured to drive the circular mouth structure to move upwards when performing the cupping operation, and correspondingly the lotus-petal-shaped mouth structure automatically moves downwards through meshing engagement.

In an embodiment, each of the meshing transmission assemblies includes a support rod, a transmission gear, a first rack and a second rack. The transmission gear is disposed on a top of the support rod through a rotating shaft, the first rack and the second rack are fixedly disposed on sidewalls of the inner cupping body and the transmission sleeve respectively, and the first rack and the second rack each are meshed with the transmission gear.

In an embodiment, the screw rod drive assembly includes a threaded groove defined on the bottom of the outer cupping body and a screw rod body disposed in the threaded groove, an end of the screw rod body extends into the interior of the outer cupping body and is rotatably connected to an installation ring fixed on a bottom of the inner cupping body, and another end of the screw rod body extends outwards and is fixedly connected with a handle.

In an embodiment, an installation part is provided on a bottom of an interior of the inner cupping body, and a top of the installation part is provided with multiple insertion needles configured to connect and fix moxa cones.

In an embodiment, a material of each of the lotus-shaped mouth and the circular mouth is brass.

In an embodiment, the fire-dragon cupping device includes a thermal insulation protective sleeve configured for easy gripping by hand and anti-scalding. The thermal insulation protective sleeve includes: a sleeve body, elastic rings disposed on upper and lower ends of the sleeve body respectively, and multiple hand straps disposed on a sidewall of the sleeve body and configured for easy gripping by hand.

In an embodiment, the fire-dragon cupping device includes first elastic column assemblies. Each of the first elastic column assemblies includes a retractable groove, a retractable rod and a spring. The retractable groove is defined in a corresponding one of the first limit strips. The retractable rod is disposed in the retractable groove, and an end of the retractable rod facing away from the retractable groove extends downwards and is fixed on the bottom of the interior of the outer cupping body. The spring is disposed on an outer wall of the retractable rod located between the corresponding one of the first limit strips and the outer cupping body.

In an embodiment, an assembly plate is disposed on the assembly ring and a drive sleeve assembly is disposed on the assembly plate. A fastening assembly is disposed between the assembly plate and the drive sleeve assembly, and the fastening assembly is configured to detachably connect the assembly plate with the drive sleeve assembly. The fire-dragon cupping device further includes a large-sized mouth. The large-sized mouth includes: a second lotus-petal-shaped mouth structure, a second circular mouth structure and second meshing transmission assemblies. Structures of the second lotus-petal-shaped mouth structure, the second circular mouth structure and the second meshing transmission assemblies are same as those of the lotus-petal-shaped mouth structure, the circular mouth structure and the meshing transmission assemblies. Second elastic column assemblies are disposed between the second circular mouth structure and the fastening assembly, the second elastic column assemblies and the first elastic column assemblies have same structures, and the second elastic column assemblies are configured to improve connection stability of the second circular mouth structure.

In an embodiment, the drive sleeve assembly includes an installation sleeve and a screw sleeve, and the installation sleeve includes a plate part distributed laterally and a threaded part distributed vertically. The screw sleeve is disposed on an outer periphery of the threaded part, and a top of the screw sleeve is rotatably connected to another assembly ring of the second lotus-petal-shaped mouth structure.

In an embodiment, the fastening assembly includes: two threaded holes, two installation strips and studs. The two threaded holes are defined on the assembly plate. The two installation strips are fixed on the plate part of the installation sleeve. Each of the two installation strips defines an installation hole therein, a top of each of the two installation strips is provided with a screw nut, and the installation hole and the screw nut are aligned with a corresponding one of the two threaded holes. The studs are configured to install and lock the installation sleeve and the assembly plate when each of the studs passes through the corresponding one of the two threaded holes and the installation hole, and is threadedly connected to the screw nut.

Compared with the related art, embodiments of the disclosure may have following beneficial effects.

1. The disclosure, through the structural arrangement of the meshing transmission assemblies, achieves the automatic switching of the vertical positions of the lotus-petal-shaped mouth structure and the circular mouth structure. After the lotus-petal-shaped mouth structure completes the scraping and moxibustion operations, the lotus-petal-shaped mouth structure is driven to descend and disengage from the skin, while the circular mouth structure ascends to a suitable position at an upper part of the outer cupping body and comes into contact with the skin. Subsequently, through the airtightness of the circular mouth structure in conjunction with the vacuum suction effect, the circular mouth structure may achieve the cupping operation including placing and retaining the cupping device on key acupoints and painful areas, thereby enhancing the therapeutic effect and thus realizing the multiple effects of scraping, moxibustion, and cupping.

2. In an embodiment of the disclosure, the thermal insulation protective sleeve serves the purpose of anti-scalding, and the hand straps of the thermal insulation protective sleeve also alleviate the decrease in finger grip strength and fatigue caused by the weight of the outer cupping body, especially when operating the large-sized cupping device with both hands.

3. The lotus-shaped mouth and the circular mouth are made of brass material. The brass has a short usage time and good effect. When the brass comes into contact with the skin, the brass promotes the self-generation of collagen of the skin. Additionally, the brass possesses antibacterial functions, including resistance to bacteria, fungi, viruses, and dust mites. Moreover, the brass is hard in texture with a relatively smooth surface. Therefore, in clinical practice, brass tools are most effective for scraping and achieving the best therapeutic results.

4. In an embodiment of the disclosure, the large-sized mouth is detachably installed on the fire-dragon cupping device in an embodiment 1, thereby improving the practicality and scope of application of the fire-dragon cupping device.

### BRIEF DESCRIPTION OF DRAWINGS

FIG. 1 illustrates a schematic structural view of a fire-dragon cupping device according to an embodiment 1 of the disclosure.

FIG. 2 illustrates a schematic exploded structural view corresponding to FIG. 1.

FIG. 3 illustrates a schematic cross-sectional view corresponding to FIG. 1.

FIG. 4 illustrates a cut-away structural view corresponding to FIG. 1.

FIG. 5 illustrates an installation structural view of the fire-dragon cupping device according to the embodiment 1 of the disclosure with an assembly plate.

FIG. 6 illustrates a schematic structural view of a fire-dragon cupping device according to an embodiment 5 of the disclosure.

FIG. 7 illustrates a front view corresponding to FIG. 6.

FIG. 8 illustrates a sectional structural view taken along the line B-B;

FIG. 9 illustrates a front view corresponding to FIG. 8.

FIG. 10 illustrates a cut-away structural view of the fire-dragon cupping device according to the embodiment 5 of the disclosure.

Description of reference signs: 1: outer cupping body; 2, lotus-petal-shaped mouth structure; 201: transmission sleeve; 202: first limit strip; 203: lotus-shaped mouth; 204: retractable groove; 205: retractable rod; 206: spring; 3: circular mouth structure; 301: connecting ring; 302: inner cupping body; 303: circular mouth; 304: second limit strip; 305: installation part; 306: insertion needle; 307: second limit groove; 4: first meshing transmission assembly; 401: support rod; 402: transmission gear; 403: first rack; 404: second rack; 405: rotating shaft; 5: screw rod drive assembly; 501: screw rod body; 502: installation ring; 503: handle; 504: threaded groove; 6: thermal insulation protective sleeve; 601: sleeve body; 602: elastic ring; 603: hand strap; 7: fastening assembly; 701: installation strip; 702: threaded hole; 703: installation hole; 704: screw nut; 705: stud; 8: installation sleeve; 801: plate part; 802: threaded part; 9: screw sleeve; 10: second lotus-petal-shaped mouth structure; 11: second circular mouth structure; 12: assembly ring; 1201: first limit groove; 13: assembly plate; 14: second meshing transmission assembly; 15: second elastic column assembly.

### DETAILED DESCRIPTION OF EMBODIMENTS

In the description of the disclosure, it should be understood that terms “length”, “width”, “up”, “down”, “front”, “back”, “left”, “right”, “vertical”, “horizontal”, “top”, “bottom”, “inside”, “outside” and other directional or positional relationships indicated are based on the directional or positional relationships shown in the accompanying drawings, only for the convenience of describing the disclosure and simplifying the description, and do not indicate or imply that the device or element referred to must have a specific orientation, be constructed and operated in a specific orientation, and therefore cannot be understood as a limitation of the disclosure. Exemplary embodiments of the disclosure will be described in detail below with reference to the accompanying drawings.

### Embodiment 1

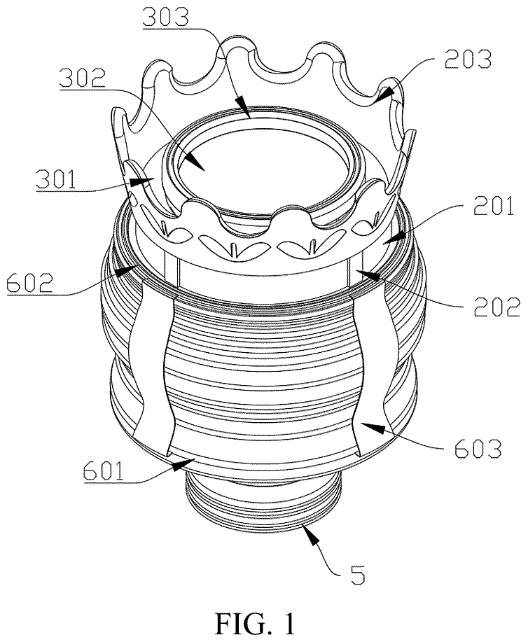

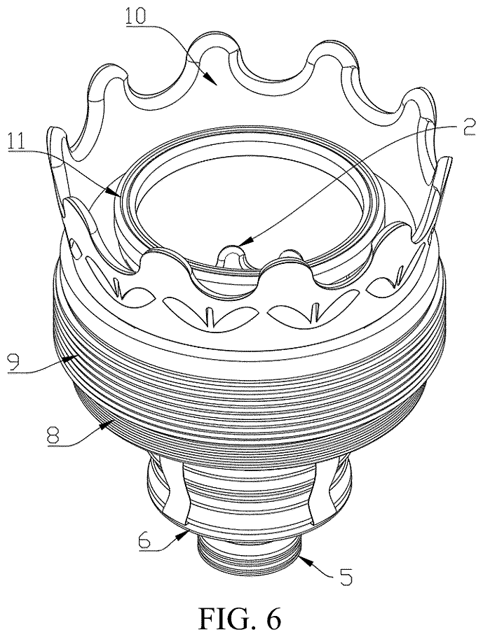

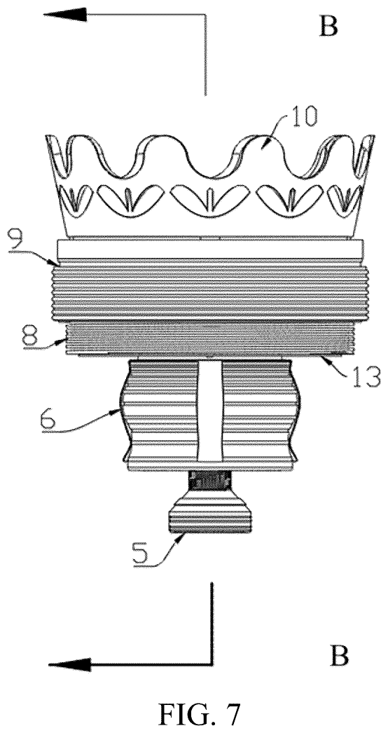

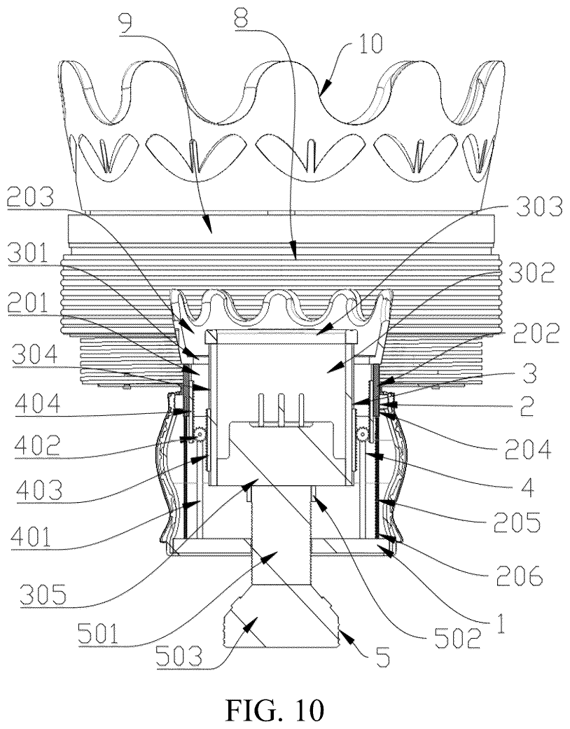

Referring to FIG. 1 to FIG. 10, an embodiment 1 of the disclosure provides following technical solutions. A fire-dragon cupping device integrated with functions of cupping and scraping includes: an outer cupping body 1, a lotus-petal-shaped mouth structure 2 disposed on the outer cupping body 1, a circular mouth structure 3, a connecting ring 301, meshing transmission assemblies 4 and a screw rod drive assembly 5. The lotus-petal-shaped mouth structure 2 includes an assembly ring 12, a transmission sleeve 201, first limit strips 202 and lotus-shaped mouth 203. The assembly ring 12 is fixedly mounted on a top of the outer cupping body 1, the first limit strips 202 are disposed around the transmission sleeve 201 and movable up and down in first limit grooves 1201 defined on an inner wall of the assembly ring 12 respectively, the lotus-shaped mouth 203 is disposed on a top of the transmission sleeve 201, and the lotus-petal-shaped mouth structure 2 is configured to perform scraping and cupping operations of the fire-dragon cupping device. The circular mouth structure 3 includes an inner cupping body 302 and a circular mouth 303, the circular mouth 303 is connected to a top of the inner cupping body 302, and the circular mouth structure 3 is configured to improve airtightness when the fire-dragon cupping device is inverted and pressed and thereby to perform a cupping operation through vacuum suction. The connecting ring 301 is disposed on an inner wall of the transmission sleeve 201, and second limit strips 304 are disposed around the inner cupping body 302 and are movable up and down in second limit grooves 307 defined on an inner wall of the connecting ring 301 respectively. Multiple first meshing transmission assemblies 4 are annularly arranged on a bottom of an interior of the outer cupping body 1. Each of the first meshing transmission assemblies 4 is disposed between the lotus-petal-shaped mouth structure 2 and the circular mouth structure 3 and configured to automatically switch vertical positions of the lotus-petal-shaped mouth structure 2 and the circular mouth structure 3. The screw rod drive assembly 5 is disposed on a bottom of the outer cupping body 1. The screw rod drive assembly 5 is configured to drive the circular mouth structure 3 to move downwards when performing the scraping and moxibustion operations, and correspondingly the lotus-petal-shaped mouth structure 2 automatically moves upwards through meshing engagement; and the screw rod drive assembly 5 is configured to drive the circular mouth structure 3 to move upwards when performing the cupping operation, and correspondingly the lotus-petal-shaped mouth structure 2 automatically moves downwards through meshing engagement.

The design highlights of the embodiment of the disclosure lies in position switching between the lotus-petal-shaped mouth structure 2 and the circular mouth structure 3, which shows the design concept of multiple uses for a single pot. It is known that current fire-dragon cupping devices in the related art have a lotus-petal-shaped structure that is integrally formed at the mouth. However, due to the mouth design of this lotus petal shape, the airtightness is insufficient, thereby failing to meet requirements for the cupping operation. Therefore, in order to enhance the practicality and functionality of a fire-dragon cupping device, this disclosure designs the fire-dragon cupping device that can be used for both cupping and scraping.

Specifically, as shown in FIG. 1 to FIG. 3, the assembly ring 12 of the lotus-petal-shaped mouth structure 2 is fixed on the top of the outer cupping body 1, an inside of the assembly ring 12 defines the first limit grooves 1201 configured to limit and install the first limit strips 202 disposed around the transmission sleeve 201. This arrangement, in combination with the circular mouth 303 disposed on the top of the transmission sleeve 201, achieves the effects of scraping, relaxing sinews and activating collaterals, and moxibustion. The inner cupping body 302 of the circular mouth structure 3 is connected to the lotus-petal-shaped mouth structure 2 through the connecting ring 301 disposed on the inner wall of the transmission sleeve 201. Specifically, the second limit strips 304 disposed around the outer periphery of the inner cupping body 302 can move in a limited manner in the second limit grooves 307 defined on the inner wall of the connecting ring 301 respectively, in combination with the circular mouth 303, to thereby achieve the vacuum cupping process.

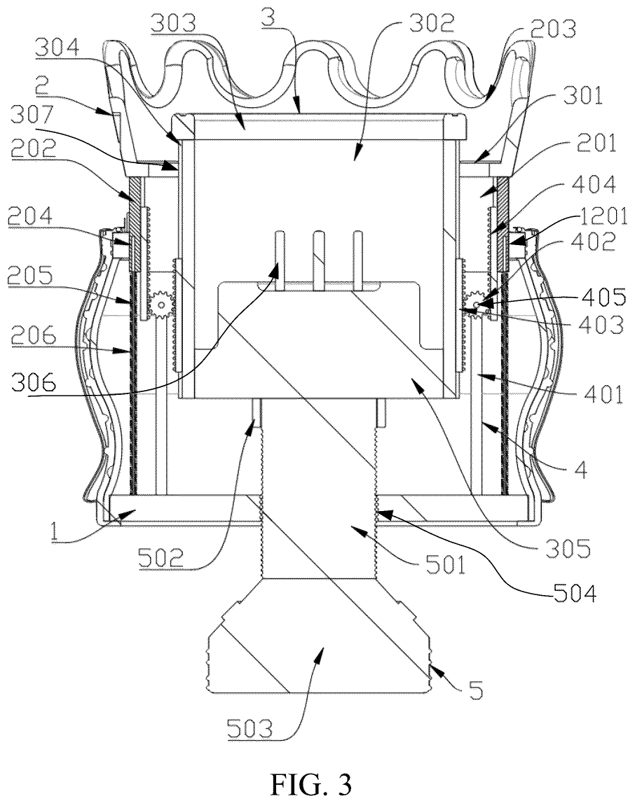

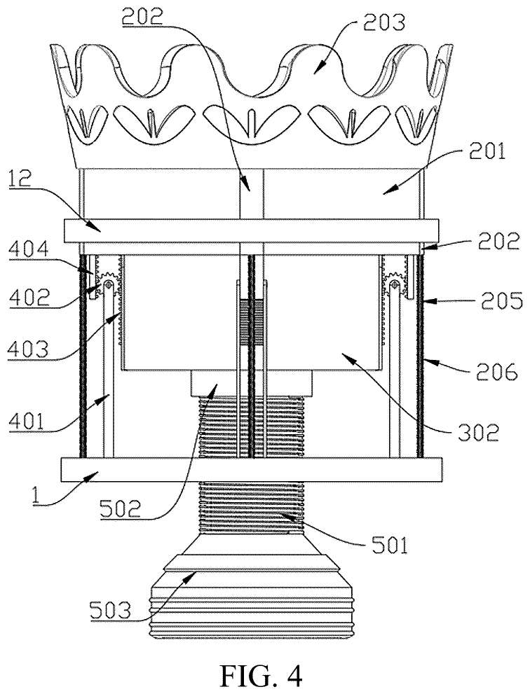

The lotus-petal-shaped mouth structure 2 and the circular mouth structure 3 achieve the position switching by the meshing transmission assemblies 4 arranged in an annular manner at the bottom of the interior of the outer cupping body 1, and the screw rod drive assembly 5 disposed at the bottom of the outer cupping body 1. Specifically, as shown in FIG. 3 to FIG. 4, when using the lotus-petal-shaped mouth structure 2 to perform the scraping and moxibustion operations, the screw rod drive assembly 5 drives the circular mouth structure 3 to move downwards, at this time, under the action of the meshing transmission assemblies 4, the lotus-petal-shaped mouth structure 2 automatically moves upwards through meshing engagement. When performing the cupping operation, the screw rod drive assembly 5 drives the circular mouth structure 3 to move upwards, and correspondingly the lotus-petal-shaped mouth structure 2 automatically moves downwards through meshing engagement. Therefore, when the lotus-petal-shaped mouth structure 2 or the t circular mouth structure 3 is needed, the required mouth is driven to move upwards to come in contact with the skin.

In an embodiment, firstly, the scraping and moxibustion operations are performed through the lotus-petal-shaped mouth structure 2, when the moxa cones therein are about to burn out, the screw rod drive assembly 5 is driven, in combination with the meshing transmission assemblies 4, to make the lotus-petal-shaped mouth structure 2 be automatically switched to the circular mouth structure 3, specifically, to make the lotus-petal-shaped mouth structure 2 move downwards and separate from the skin, and the circular mouth structure 3 move upwards to a suitable position on the upper part of the outer cupping body 1 to be in contact with the skin. Subsequently, the airtightness of the circular mouth structure 3, in combination with vacuum suction effect, is used to achieve the cupping operation including placing and retaining the cupping device on key acupoints and painful areas, thereby enhancing the therapeutic effect and thus realizing the multiple effects of scraping, moxibustion and cupping.

The disclosure, by switching the positions of the lotus-petal-shaped mouth structure 2 and the circular mouth structure 3, and combining the structural characteristics of the lotus-petal-shaped mouth structure 2 and the circular mouth structure 3, the fire-dragon cupping device simultaneously possesses the multiple therapeutic functions of scraping, moxibustion, and cupping. The design concept of the fire-dragon cupping device, which allows for multiple uses with a single pot, enhances the therapeutic effect of the fire-dragon cupping device and improves the practicality of the fire-dragon cupping device.

In an embodiment, each of the meshing transmission assemblies 4 includes a support rod 401, a transmission gear 402, a first rack 403 and a second rack 404. The transmission gear 402 is disposed on a top of the support rod 401 through a rotating shaft 405, the first rack 403 and the second rack 404 are fixedly disposed on sidewalls of the inner cupping body 302 and the transmission sleeve 201 respectively, and the first rack 403 and the second rack 404 each are meshed with the transmission gear 402.

As shown in FIG. 3 to FIG. 4, the first rack 403 and the second rack 404 are fixedly disposed on the sidewalls of the inner cupping body 302 and the transmission sleeve 201 respectively, and meshed with the transmission gear 402 individually. Therefore, when the inner cupping body 302 of the circular mouth structure 3 moves upwards or downwards under the action provided by the screw rod drive assembly 5, the transmission sleeve 201 of the lotus-petal-shaped mouth structure 2 simultaneously moves downwards or upwards driven by the transmission gear 402, thereby achieving the position switching process between the lotus-petal-shaped mouth structure 2 and the circular mouth structure 3.

In an embodiment, the screw rod drive assembly 5 includes a threaded groove 504 defined on the bottom of the outer cupping body 1 and a screw rod body 501 disposed in the threaded groove 504, an end of the screw rod body 501 extends into the interior of the outer cupping body 1 and is rotatably connected to an installation ring 502 fixed on a bottom of the inner cupping body 302, and another end of the screw rod body 501 extends outwards and is fixedly connected with a handle 503.

As shown in FIG. 3 to FIG. 4, when rotating the handle 503, the spiral lifting and lowering process of the screw rod body 501 within the threaded groove 504 on the bottom of the outer cupping body 1 is achieved. In conjunction with the installation ring 502 that is fixed on the bottom of the inner cupping body 302 and rotatably connected to the screw rod body 501, it is known that the inner cupping body 302 is limitedly connected to the second limit grooves 307 of the connecting ring 301 through the second limit strips 304 disposed around the outer periphery of the inner cupping body 302, and thus, when the screw rod body 501 undergoes spiral lifting and lowering, the limited lifting and lowering process of the inner cupping body 302 can be achieved.

In an embodiment, a bottom of an interior of the inner cupping body 302 is provided with an installation part 305, and a top of the installation part 305 is provided with multiple insertion needles 306 configured to insert and fix moxa cones, as shown in FIG. 3.

### Embodiment 2

In an embodiment of the disclosure, a material of each of the lotus-shaped mouth 203 and the circular mouth 303 is brass.

The brass is a good conductor of qi (qi refers to a vital energy within the human body in traditional Chinese medicine, which runs through various parts of the body and supports life activities), and the scraping requires the arrival of qi. When it is necessary to use manual techniques to guide heat into the qi entering vessel of the patient, the brass has a short usage time and provides good results. Copper (not a heavy metal) is one of the 11 essential elements for the human body. When the copper comes into contact with the skin, it can promote the skin's natural production of collagen. Additionally, the copper has antibacterial properties, being effective against bacteria, fungi, viruses, and dust mites. For example, the University of Southampton in the UK conducted an experiment on the bactericidal ability of copper, and found that bacteria on a copper plate were completely eradicated within nine minutes, whereas the bacteria could survive for several weeks on a stainless steel plate. Moreover, the brass is hard in texture and has a relatively smooth surface. Therefore, in clinical practice, tools made of brass are most effective for scraping, as they are more likely to produce desired marks (i.e., Sha in traditional Chinese medicine) and achieve the best therapeutic results. Since gliding cupping is a form of scraping, both the circular mouth 303 and the lotus-shaped mouth 203 are made of the brass.

### Embodiment 3

In an embodiment of the disclosure, the fire-dragon cupping device further includes a thermal insulation protective sleeve 6 configured for easy gripping by hand and anti-scalding. The thermal insulation protective sleeve 6 includes: a sleeve body 601, elastic rings 602 and multiple hand straps 603. The elastic rings 602 are disposed on upper and lower ends of the sleeve body 601 respectively, and the multiple hand straps 603 are disposed on a sidewall of the sleeve body 601 and configured for easy gripping by hand.

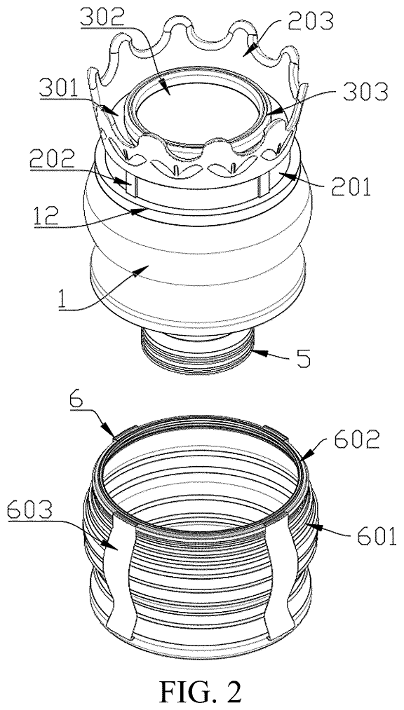

The thermal insulation protective sleeve 6 in the embodiment includes the elastic rings 602 disposed on the upper and lower ends of the sleeve body 601, which is configured to make the thermal insulation protective sleeve 6 cover the outer cupping body 1 better to guarantee the sleeved connection stability of the thermal insulation protective sleeve 6. The multiple hand straps 603 are disposed on the sidewall of the sleeve body 601 and for example, a number of the multiple hand straps 603 is four. As shown in FIG. 2, hands can pass through the hand straps 603 to directly lift the outer cupping body 1, which is time-saving and labor-saving, and alleviates the decrease in finger grip strength and fatigue caused by the weight of the outer cupping body 1, especially when operating a large-sized cupping device with both hands.

In addition, as shown in FIG. 2, the sleeve body 601 is made of an elastic material and provided with multiple protruding strips on an inner wall of the sleeve body 601, and the outer surface of the outer cupping body 1 defines grooves corresponding to the protruding strips. This interlocking design of protrusion strips and grooves further enhances the stability of the outer cupping body 1 being wrapped by the sleeve body 601.

### Embodiment 4

In an embodiment, the fire-dragon cupping device further includes first elastic column assemblies. Each of the first elastic column assemblies includes a retractable groove (204), a retractable rod (205) and a spring (206). The retractable groove (204) is defined in a corresponding one of the first limit strips (202). The retractable rod (205) is disposed in the retractable groove (204). An end of the retractable rod (205) facing away from the retractable groove (204) extends downwards and is fixed on the bottom of the interior of the inner cupping body (1). The spring (206) is disposed on an outer wall of the retractable rod (205) located between the corresponding one of the first limit strips (202) and the outer cupping body (1).

As shown in FIG. 3 to FIG. 4, the structural design of the first elastic column assemblies ensures better stability during the lifting and lowering of the lotus-petal-shaped mouth structure (2) by the meshing transmission assemblies (4).

### Embodiment 5

In an embodiment, the assembly ring 12 is provided with an assembly plate 13, and the assembly plate 13 is provided with a drive sleeve assembly. A fastening assembly 7 is disposed between the assembly plate 13 and the drive sleeve assembly, and the fastening assembly 7 is configured to detachably connect the assembly plate 13 with the drive sleeve assembly. The fire-dragon cupping device further includes a large-sized mouth; the large-sized mouth includes: a second lotus-petal-shaped mouth structure 10, a second circular mouth structure 11 and second meshing transmission assemblies 14; and structures of the second lotus-petal-shaped mouth structure 10, the second circular mouth structure 11 and the second meshing transmission assemblies 14 are same as structures of the lotus-petal-shaped mouth structure 2, the circular mouth structure 3 and the meshing transmission assemblies 4. Second elastic column assemblies 15 are disposed between the second circular mouth structure 11 and the fastening assembly 7, the second elastic column assemblies 15 and the first elastic column assemblies have same structures, and the second elastic column assemblies 15 are configured to improve connection stability of the second circular mouth structure 11.

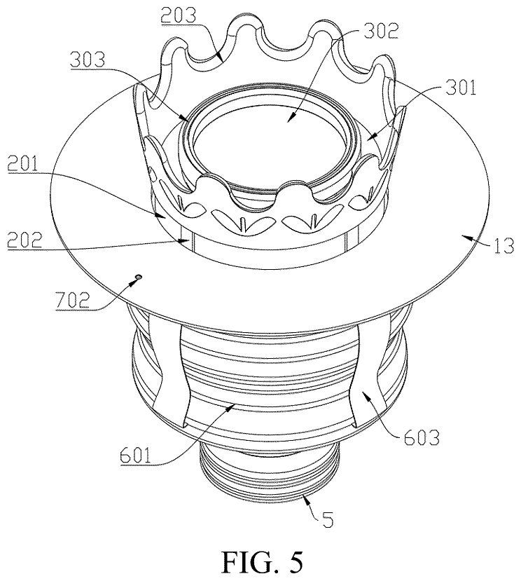

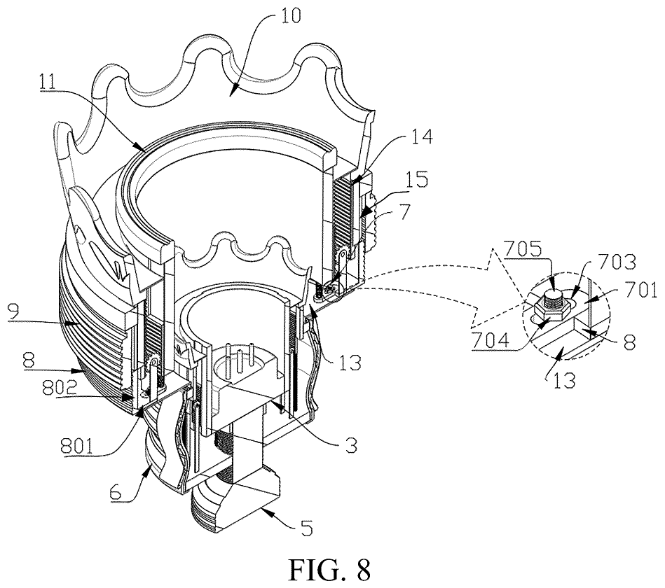

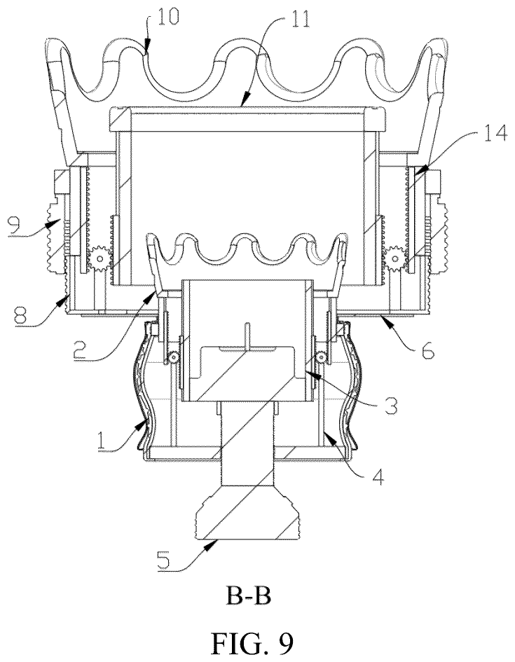

In the embodiment, as shown in FIG. 5 to FIG. 7, the large-sized mouth includes the second lotus-petal-shaped mouth structure 10, the second circular mouth structure 11 and the second meshing transmission assemblies 14. The structures of the second lotus-petal-shaped mouth structure 10, the second circular mouth structure 11 and the second meshing transmission assemblies 14 are same as the structures of the lotus-petal-shaped mouth structure 2, the circular mouth structure 3 and the meshing transmission assemblies 4. Therefore, in conjunction with FIG. 9, specific structures of the second lotus-petal-shaped mouth structure 10, the second circular mouth structure 11 and the second meshing transmission assemblies 14 can refer to the detailed explanations of the t lotus-petal-shaped mouth structure 2, the circular mouth structure 3 and the meshing transmission assemblies 4 described above. The large-sized mouth is set based on the fire-dragon cupping device in the embodiment 1, and is detachably installed on the fire-dragon cupping device in the embodiment 1, thereby further improving the practicality and scope of application of the fire-dragon cupping device, as shown in FIG. 8.

As shown in FIG. 9, the drive sleeve assembly as a drive structure of the second lotus-petal-shaped mouth structure 10 is configured to, in conjunction with the second meshing transmission assemblies 14 disposed between the second lotus-petal-shaped mouth structure 10 and the second circular mouth structure 11, achieve the opposite operating processes of the second lotus-petal-shaped mouth structure 10 and the second circular mouth structure 11.

The second elastic column assemblies 15 serve as a stabilizing structure during the operating of the second circular mouth structure 11.

In an embodiment, the drive sleeve assembly includes an installation sleeve 8 and a screw sleeve 9, and the installation sleeve 8 includes a plate part 801 distributed laterally and a threaded part 802 distributed vertically. The screw sleeve 9 is disposed on an outer periphery of the threaded part 802, and a top of the screw sleeve 9 is rotatably connected to another assembly ring 12 of the second lotus-petal-shaped mouth structure 10.

As shown in FIG. 8 to FIG. 9, when it is needed to switch positions of the second lotus-petal-shaped mouth structure 10 and the second circular mouth structure 11, the plate part 801 of the installation sleeve 8 is fixed on the assembly plate 13 through the fastening assembly 7, the threaded part 802 of the installation sleeve 8 is threadedly connected to the screw sleeve 9, and the screw sleeve 9 is rotatably connected to the another assembly ring 12 of the second lotus-petal-shaped mouth structure 10, therefore, when rotating the screw sleeve 9 to move upwards, the another assembly ring 12 of the second lotus-petal-shaped mouth structure 10 is driven to move upwards to achieve the lifting process of the second lotus-petal-shaped mouth structure 10; meanwhile, in conjunction with the action of the second meshing transmission assemblies 14, the lowering process of the second circular mouth structure 11 is thereby achieved. Similarly, when the screw sleeve 9 moves downwards, the second lotus-petal-shaped mouth structure 10 and the second circular mouth structure 11 moves in the opposite direction.

Furthermore, the fastening assembly 7 includes two threaded holes 702, two installation strips 701 and studs 705. The two threaded holes 702 are defined on the assembly plate 13. The two installation strips 701 are fixed on the plate part 801 of the installation sleeve 8. Each of the two installation strips 701 defines an installation hole 703 therein, a top of each of the two installation strips 701 is provided with a screw nut 704, and the installation hole 703 and the screw nut 704 are aligned with a corresponding one of the two threaded holes 702. The studs 705 are configured to install and lock the installation sleeve 8 and the assembly plate 13 when each of the studs 705 passes through the corresponding one of the two threaded holes 702 and the installation hole 703, and is threadedly connected to the screw nut 704.

The two threaded holes 702 are defined on two sides of the assembly plate 13, as shown in FIG. 5. The two installation strips 701 are disposed on the installation sleeve 8 corresponding to the two threaded holes 702, as shown in FIG. 8. The screw nut 704 disposed on a top and middle part of each installation strip 701 is aligned with the corresponding threaded hole 702. Therefore, when each stud 705 passes through the corresponding threaded hole 702 and the installation hole 703 and is threadedly connected to the screw nut 704, the studs 705 are configured to install and lock the large-sized mouth onto the fire-dragon cupping device in the embodiment 1.

In the disclosure, unless otherwise specified and limited, the terms “installation”, “connection”, and “fixation”, etc. should be broadly understood, for example, it can be a fixed connection, a detachable connection, or an integral structure; it can be a direct connection or an indirectly connection through an intermediate medium, for example, it can be achieved through the combination of plugs and buckles, or through bolt connections.

Based on the embodiments of the disclosure, other embodiments obtained by those skilled in the art without creative labor are within the scope of protection of the disclosure. In addition, all the connections mentioned herein do not simply refer to the direct connection of components, but rather indicate that, depending on specific implementation scenarios, more optimal connection structures can be formed by adding or reducing auxiliary connection parts.

The specific description of the disclosure in the above embodiments is only for further explanation of the disclosure and cannot be understood as limiting the scope of protection of the disclosure. Technical engineers in this field have made some non-essential improvements and adjustments to the disclosure based on the content of the disclosure, all of which fall within the scope of protection of the disclosure.

What is claimed is:

1. A fire-dragon cupping device integrated with functions of cupping and scraping, comprising:
an outer cupping body (1);
a lotus-petal-shaped mouth structure (2), disposed on the outer cupping body (1), wherein the lotus-petal-shaped mouth structure (2) comprises an assembly ring (12), a transmission sleeve (201), first limit strips (202), and lotus-shaped mouth (203); the assembly ring (12) is fixedly mounted on a top of the outer cupping body (1), the first limit strips (202) are disposed around the transmission sleeve (201) and movable up and down in first limit grooves (1201) defined on an inner wall of the assembly ring (12) respectively, the lotus-shaped mouth (203) is disposed on a top of the transmission sleeve (201), and the lotus-petal-shaped mouth structure (2) is configured to perform scraping and moxibustion operations of the fire-dragon cupping device;
a circular mouth structure (3), wherein the circular mouth structure (3) comprises an inner cupping body (302) and a circular mouth (303), the circular mouth (303) is connected to a top of the inner cupping body (302), and the circular mouth structure (3) is configured to improve airtightness when the fire-dragon cupping device is inverted and pressed and thereby to perform a cupping operation through vacuum suction;
a connecting ring (301), disposed on an inner wall of the transmission sleeve (201), wherein the inner cupping body (302) is provided with second limit strips (304) arranged therearound, and the second limit strips (304) are movable up and down in second limit grooves (307) defined on an inner wall of the connecting ring (301) respectively;
meshing transmission assemblies (4), annularly arranged on a bottom of an interior of the outer cupping body (1), wherein each of the meshing transmission assemblies (4) is disposed between the lotus-petal-shaped mouth structure (2) and the circular mouth structure (3) and configured to automatically switch vertical positions of the lotus-petal-shaped mouth structure (2) and the circular mouth structure (3); and
a screw rod drive assembly (5), disposed on a bottom of the outer cupping body (1), wherein the screw rod drive assembly (5) is configured to drive the circular mouth structure (3) to move downwards when performing the scraping and moxibustion operations, and correspondingly the lotus-petal-shaped mouth structure (2) automatically moves upwards through meshing engagement, and configured to drive the circular mouth structure (3) to move upwards when performing the cupping operation, and correspondingly the lotus-petal-shaped mouth structure (2) automatically moves downwards through meshing engagement;
wherein each of the meshing transmission assemblies (4) comprises a support rod (401), a transmission gear (402), a first rack (403) and a second rack (404); the transmission gear (402) is disposed on a top of the support rod (401) through a rotating shaft (405), the first rack (403) and the second rack (404) are fixedly disposed on sidewalls of the inner cupping body (302) and the transmission sleeve (201) respectively, and the first rack (403) and the second rack (404) each are meshed with the transmission gear (402);
wherein the screw rod drive assembly (5) comprises a threaded groove (504) defined on the bottom of the outer cupping body (1) and a screw rod body (501) disposed in the threaded groove (504), an end of the screw rod body (501) extends into the interior of the outer cupping body (1) and is rotatably connected to an installation ring (502) fixed on a bottom of the inner cupping body (302), and another end of the screw rod body (501) extends outwards and is fixedly connected with a handle (503).

2. The fire-dragon cupping device integrated with functions of cupping and scraping as claimed in claim 1, wherein an installation part (305) is provided on a bottom of an interior of the inner cupping body (302), and a top of the installation part (305) is provided with insertion needles (306) configured to connect and fix moxa cones.

3. The fire-dragon cupping device integrated with functions of cupping and scraping as claimed in claim 1, wherein a material of each of the lotus-shaped mouth (203) and the circular mouth (303) is brass.

4. The fire-dragon cupping device integrated with functions of cupping and scraping as claimed in claim 1, comprising: an thermal insulation protective sleeve (6) configured for easy gripping by hand and anti-scalding; wherein the thermal insulation protective sleeve (6) comprises:
a sleeve body (601),
elastic rings (602), disposed on upper and lower ends of the sleeve body (601) respectively, and
a plurality of hand straps (603), disposed on a sidewall of the sleeve body (601) and configured for easy gripping by hand.

5. The fire-dragon cupping device integrated with functions of cupping and scraping as claimed in claim 1, further comprising: first elastic column assemblies, wherein each of the first elastic column assemblies comprises:
a retractable groove (204), defined in a corresponding one of the first limit strips (202);
a retractable rod (205), disposed in the retractable groove (204), wherein an end of the retractable rod (205) facing away from the retractable groove (204) extends downwards and is fixed on the bottom of the interior of the outer cupping body (1); and
a spring (206), disposed on an outer wall of the retractable rod (205) located between the corresponding one of the first limit strips (202) and the outer cupping body (1).

6. The fire-dragon cupping device integrated with functions of cupping and scraping as claimed in claim 5, wherein an assembly plate (13) is disposed on the assembly ring (12), and a drive sleeve assembly is disposed on the assembly plate (13);
wherein a fastening assembly (7) is disposed between the assembly plate (13) and the drive sleeve assembly, and the fastening assembly (7) is configured to detachably connect the assembly plate (13) with the drive sleeve assembly;
wherein the fire-dragon cupping device further comprises a large-sized mouth; the large-sized mouth comprises: a second lotus-petal-shaped mouth structure (10), a second circular mouth structure (11) and second meshing transmission assemblies (14); and structures of the second lotus-petal-shaped mouth structure (10), the second circular mouth structure (11) and the second meshing transmission assemblies (14) are same as those of the lotus-petal-shaped mouth structure (2), the circular mouth structure (3) and the meshing transmission assemblies (4); and
wherein second elastic column assemblies (15) are disposed between the second circular mouth structure (11) and the fastening assembly (7), the second elastic column assemblies (15) and the first elastic column assemblies have same structures, and the second elastic column assemblies (15) are configured to improve connection stability of the second circular mouth structure (11).

7. The fire-dragon cupping device integrated with functions of cupping and scraping as claimed in claim 6, wherein the drive sleeve assembly comprises:
an installation sleeve (8), comprising a plate part (801) distributed laterally and a threaded part (802) distributed vertically; and
a screw sleeve (9), disposed on an outer periphery of the threaded part (802), and a top of the screw sleeve (9) being rotatably connected to another assembly ring (12) of the second lotus-petal-shaped mouth structure (10).

8. The fire-dragon cupping device integrated with functions of cupping and scraping as claimed in claim 7, wherein the fastening assembly (7) comprises:
two threaded holes (702), defined on the assembly plate (13);
two installation strips (701), fixed on the plate part (801) of the installation sleeve (8), wherein each of the two installation strips (701) defines an installation hole (703) therein, a top of each of the two installation strips (701) is provided with a screw nut (704), and the installation hole (703) and the screw nut (704) are aligned with a corresponding one of the two threaded holes (702); and
studs (705), configured to install and lock the installation sleeve (8) and the assembly plate (13) when each of the studs (705) passes through the corresponding one of the two threaded holes (702) and the installation hole (703), and is threadedly connected to the screw nut (704).
